SEQUENCE LISTING (2 sequences)

SEQ ID NO: 1 - protein, 685 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 2 - DNA, 2055 bp, Severe acute respiratory syndrome coronavirus 2
  source 1..2055: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgt

(19) United States
(12) Patent Application Publication
(10) Pub. No.: US 2025/0090516 A1
(43) Pub. Date: Mar. 20, 2025

(54) DRUG FOR TREATING AFTEREFFECTS OF NOVEL CORONAVIRUS INFECTION

(71) Applicant: THE JIKEI UNIVERSITY, Tokyo (JP)

(72) Inventors: Kazuhiro KONDO, Tokyo (JP); Naomi OKA, Tokyo (JP)

(21) Appl. No.: 18/728,041

(22) PCT Filed: Dec. 6, 2022
(86) PCT No.: PCT/JP2022/044828

§ 371 Date: Jul. 10, 2024

(30) Foreign Application Priority Data
Jan. 14, 2022 (JP) 2022-004245

(51) Int. Cl. A61K 31/445; A01K 67/027; A61P 25/24; C07K 14/005; C12N 15/86

(52) U.S. Cl. CPC A61K 31/445; A01K 67/027; A61P 25/24; C07K 14/005; C12N 15/86; A01K 2267/0337; C12N 2710/16143; C12N 2770/20022

(57) ABSTRACT

Provided is a therapeutic agent for novel coronavirus infection sequelae. The therapeutic agent for novel coronavirus infection sequelae contains an acetylcholine receptor agonist as an active ingredient.

### TECHNICAL FIELD

The present invention relates to a therapeutic agent for a sequela of a novel coronavirus infection, a method for screening a therapeutic agent for a sequela of a novel coronavirus infection, and a method for producing a novel coronavirus infection sequela model animal.

### BACKGROUND ART

The acute respiratory disease COVID-19 (so-called novel coronavirus infection), caused by the SARS-COV-2 virus, is a serious infectious disease that has infected a large number of people worldwide.

COVID-19 is known to not only causes severe disease during the acute phase and thus leads to death in some patients but also frequently cause a sequela (hereinafter referred to as “novel coronavirus infection sequela”) during the recovery phase. It is believed that, among the novel coronavirus infection sequelae, particularly common sequelae are fatigue, depressive symptoms, and olfactory dysfunctions. In addition, the novel coronavirus infection sequela can occur in patients whose symptoms during the acute phase are not severe. It is therefore believed that the sequela are likely to occur even in cases where severe symptoms are mitigated by vaccination.

As the term suggests, a novel coronavirus infection sequela is an aftereffect of a viral infection. Therefore, at the time when the sequela occurs, the proliferation of the causative virus, SARS-COV-2 virus, is not taking place. Consequently, the administration of a drug that can suppress viral proliferation after the onset of a novel coronavirus infection sequela is not expected to be effective against the sequela. Thus, it is believed that a therapeutic agent for a novel coronavirus infection sequela needs to repair tissue damage caused by the SARS-COV-2 infection.

There are currently no effective treatment for a novel coronavirus infection sequela. Consequently, unproven folk remedies are widespread, leading to health damage.

### CITATION LIST

### Non-patent Literature

### Non-patent Literature 1

Do vaccines protect against long COVID? What the data say. Heidi Ledford. Nature. November 2021; 599 (7886): 546-548.

### Non-patent Literature 2

Promotion of non-evidence-based therapeutics within patient-led Long COVID support groups. Vicky van der Togt et al. Nat Med. December 2021; 27 (12): 2068-2069.

### Non-patent Literature 3

The lesson of ivermectin: meta-analyses based on summary data alone are inherently unreliable. Jack M Lawrence et al. Nat Med. November 2021; 27 (11): 1853-1854.

### SUMMARY OF INVENTION

### Technical Problem

There are demands for therapeutic agents for novel coronavirus infection sequelae. However, no effective therapeutic agents exist. In order to conduct research on novel coronavirus infection sequelae to develop a therapeutic agent, it is important to have animal models that exhibit the symptoms of novel coronavirus infection sequelae. However, the focus of the research on the existing animal models relating to the novel coronavirus has been on the infection prevention and acute symptoms. Therefore, the development of animal models for the research on sequelae is insufficient.

An object of an aspect of the present invention is to provide a therapeutic agent for the sequelae of the novel coronavirus infection.

### Solution to Problem

The inventors of the present invention conducted diligent studies in order to attain the object and, as a result, discovered for the first time that the S1 region of the Spike protein of the SARS-COV-2 virus is the causative protein of the novel coronavirus infection sequelae. Thus, the inventors of the present invention completed an animal model that exhibits the symptoms of novel coronavirus infection sequelae. Through research on novel coronavirus infection sequelae using the novel coronavirus infection sequela model animals, the inventors of the present invention discovered for the first time that acetylcholine in the brain is decreased in novel coronavirus infection sequelae and that the novel coronavirus infection sequelae can be treated or prevented by receptor agonists.

That is, an aspect of the present invention is a therapeutic agent for a sequela of a novel coronavirus infection, the therapeutic agent including an acetylcholine receptor agonist as an active ingredient.

In addition, an aspect of the present invention is a method for screening a therapeutic agent for a sequela of a novel coronavirus infection, said method including: administering a test substance into a novel coronavirus infection sequela model animal obtained by expressing SARS-CoV-2 S1 protein in a non-human mammal; and
evaluating a change in a novel coronavirus-related symptom of the model animal before and after administration of the test substance.

In addition, an aspect of the present invention is a method for producing a novel coronavirus infection sequela model animal, including expressing SARS-COV-2 S1 protein in a non-human mammal with use of a SARS-COV-2 S1 protein expression vector.

### Advantageous Effects of Invention

With an aspect of the present invention, it is possible to provide a therapeutic agent for a novel coronavirus infection sequela.

### BRIEF DESCRIPTION OF DRAWINGS

FIG. 1 is a view schematically illustrating the structure of SARS-COV-2 Spike protein.

FIG. 2 is a view showing the results of Examples and the results of measuring intracellular calcium concentrations in 3T3 cells and A549 cells transfected with S1 protein expression plasmid or control plasmid.

FIG. 3 is a view showing the results of Examples and the results of measuring intracellular calcium concentrations in 3T3 cells and A549 cells infected with S1 protein-expressing adenovirus or control adenovirus.

FIG. 4 is a view showing the results of Examples and the results of a 10% weight-loaded forced swimming test on control mice and S1 protein-expressing mice (S1 mice).

FIG. 5 is a view showing the results of Examples and the results of a tail suspension test on control mice and S1 mice.

FIG. 6 is a view showing the results of Examples and the results of analyzing the gene expression levels of calbindin in the olfactory bulbs of control mice and S1 mice after LPS administration.

FIG. 7 is a view showing the results of Examples and the results of analyzing the gene expression levels of calbindin in the brains of control mice and S1 mice after LPS administration.

FIG. 8 is a view showing the results of Examples and damage to cholinergic neurons in control mice and S1 mice.

FIG. 9 is a view showing the results of Examples and damage to cholinergic neurons in control mice and S1 mice.

FIG. 10 is a diagram for describing the administration scheme of donepezil.

FIG. 11 is a view showing the results of Examples and the results of a 10% weight-loaded forced swimming test on control mice and S1 mice after administration of donepezil.

FIG. 12 is a view showing the results of Examples and the results of a tail suspension test on control mice and S1 mice after administration of donepezil.

FIG. 13 is a view showing the results of Examples and the results of analyzing the gene expression levels of interleukin-6 (IL-6), tumor necrosis factor (TNFα), and chemokine CC motif ligand 2 (CCL2) in control mice and S1 mice after administration of donepezil.

FIG. 14 is a view showing the results of Examples and the results of analyzing the gene expression levels of interleukin-1 beta (IL-1ß) and interleukin-6 (IL-6) in control mice and S1 mice after administration of PNU282987.

FIG. 15 is a view showing the results of Examples and the results of analyzing the gene expression levels of ZFP36 in control mice and S1 mice after administration of PNU282987.

### Description of Embodiments

### 1. Therapeutic Agent

### Features

A therapeutic agent in accordance with an aspect of the present invention for the sequelae of the novel coronavirus infection (COVID-19) (hereinafter referred to as “COVID-19 sequelae”) is a pharmaceutical composition for use in the treatment or prevention of COVID-19 sequelae, and contains an acetylcholine receptor agonist as an active ingredient. This allows for the treatment or prevention of COVID-19 sequelae in patients infected with the novel coronavirus. Therefore, it is possible to contribute to “Good Health and Well-Being”, which is Goal 3 of the sustainable development goals (SDGs). Hereinafter, for convenience of explanation, the therapeutic agent in accordance with an aspect of the present invention for the COVID-19 sequelae may be simply referred to as “therapeutic agent”.

In the present specification, “treat” includes an act of curing or alleviating the symptoms of COVID-19 sequelae and an act of suppressing the worsening of the symptoms of COVID-19 sequelae in a subject into which the therapeutic agent in accordance with an aspect of the present invention has been administered (hereinafter simply referred to as “administration subject”). In an aspect of the present invention, “prevent” includes an act of suppressing or delaying the onset of the symptoms of COVID-19 sequelae in an administration subject.

If the administration subject is exhibiting a symptom of a COVID-19 sequela, the therapeutic agent in accordance with an aspect of the present invention is used for the treatment of the COVID-19 sequela. If the administration subject is not exhibiting a symptom of a COVID-19 sequela, the therapeutic agent in accordance with an aspect of the present invention is used for the prevention of the COVID-19 sequela.

In the present specification, while the definitions of “treatment” and “prevent” are as discussed above, the mechanism of action of the therapeutic agent in accordance with an aspect of the present invention is the same in either case. Therefore, the “therapeutic agent” can be replaced with “preventive agent”. That is, if a patient infected with the novel coronavirus is not exhibiting any symptoms of COVID-19 sequelae, “treatment” means “prevention”.

It should be noted here that “COVID-19 sequelae” in general refers to the overall symptoms that appear during the recovery phase after the infection with the novel coronavirus and persists for several weeks to several months thereafter. However, the definition thereof is not established. COVID-19 sequelae are also sometimes referred to as Long COVID. In the present specification, “COVID-19 sequelae” refers to symptoms related to brain and nerve dysfunctions such as fatigue, depressive symptoms, olfactory dysfunctions, memory impairment, decreased concentration, decreased thinking ability, and decreased cognitive function which occur particularly frequently among the aforementioned symptoms.

In the present specification, one aspect of “COVID-19 sequelae” is novel coronavirus-related fatigue. In the present specification, another aspect of “COVID-19 sequelae” is a novel coronavirus-related depressive symptom. In the present specification, another aspect of “COVID-19 sequelae” is a novel coronavirus-related olfactory dysfunction. In the present specification, another aspect of “COVID-19 sequelae” is novel coronavirus-related memory impairment.

In the present specification, “novel coronavirus-related depressive symptom” refers to symptoms of a disease generally diagnosed as depression. Examples of the symptoms include not only those used in the diagnosis of major depressive disorder according to DSM-5, such as depressed mood, loss of interest or pleasure, appetite disturbance, overeating, sleep disorders, hypersomnia, psychomotor agitation or retardation, easy fatigability, feeling of worthlessness or guilt, decreased concentration, decreased thinking ability, and suicidal ideation, but also depressive states which exhibit symptoms highly frequently observed in depression patients, such as anxiety, memory impairment, aging, pain, and chronic pain. That is, in the present specification, “novel coronavirus-related depressive symptom” is a concept that is not limited to major depressive disorder but includes (i) diseases that exhibit the aforementioned depressive symptoms such as stress-related depression, depressive states of bipolar disorder, and atypical depression and (ii) symptoms of depressive states in other diseases.

In the present specification, “novel coronavirus-related fatigue” refers to a persistent or chronic phenomenon accompanied by diseases of the central nervous system and the like and means pathological fatigue, which is characterized mainly by loss of interest or pleasure, sleep disorder, psychomotor agitation or retardation, easy fatigability, decreased concentration, and decreased thinking ability. “Fatigue” can also be expressed as a feeling of tiredness or lethargy.

In the present specification, “pathological fatigue” refers to persistent or chronic fatigue associated with novel coronavirus infection. “Pathological fatigue” is distinguished from “physiological fatigue”, which refers to a temporary qualitative or quantitative decline in physical and mental work performance observed in healthy individuals when subjected to a continuous physical or mental load.

In the present specification, “novel coronavirus-related olfactory dysfunction” refers to a symptom or a disease that causes some abnormality in the olfactory sense, that is, the sense of “smell”. “Novel coronavirus-related olfactory dysfunction” is also referred to as “olfactory abnormality”. The main symptoms include hyposmia, parosmia, anosmia, and hyperosmia.

In the present specification, “novel coronavirus-related memory impairment” refers to a condition in which all or part of the four processes that constitute memory, that is, memorization, retention, retrieval, and recognition, do not function normally.

In the present specification, “symptom” refers to phenomena and conditions (abnormalities) that appear in patients due to the effect of novel coronavirus-related diseases, and is a concept that includes subjective symptoms, which are abnormalities that a patient can perceive as symptoms of the disease, and objective symptoms, which are abnormalities that can be objectively confirmed as symptoms of the disease through, for example, medical examination or a test by a doctor. It should be noted that in the animal models discussed later, changes in behavior and objective findings from tests, for example, are collectively referred to as symptoms.

The inventors of the present invention studied the brain changes in COVID-19 sequelae with use of COVID-19 sequela model animals. As a result, the inventors of the present invention found for the first time that, in the brains of COVID-19 sequelae, the number of cholinergic neurons, which are choline acetyltransferase (ChAT) positive cells in the medial septum (MS) and the diagonal band (DB) of Broca of the basal forebrain, is decreased.

This result suggests that the amount of acetylcholine is decreased in COVID-19 sequelae and suggests that acetylcholine receptor agonists are suitable as therapeutic agents for COVID-19 sequelae.

Through research on COVID-19 sequelae with use of COVID-19 sequela model animals, the inventors of the present invention for the first time discovered that the administration of acetylcholine receptor agonists improves the depressive symptoms and fatigue symptoms exhibited by the COVID-19 sequela model animals. That is, the inventors of the present invention for the first time discovered that the administration of acetylcholine receptor agonists can treat or prevent COVID-19 sequelae.

Conventionally, it has been common technical knowledge that the administration of acetylcholine receptor agonists increases the amount of acetylcholine in the brain and thus leads to the onset of depressive symptoms (for example, Reference Literature 1: Cholinergic regulation of mood: from basic and clinical studies to emerging therapeutics. Stephanie C Dulawa and David S Janowsky. Mol Psychiatry. May 2019; 24 (5): 694-709.; and Reference Literature 2: Maintenance treatment of depression in old age: a randomized, double-blind, placebo-controlled evaluation of the efficacy and safety of donepezil combined with antidepressant pharmacotherapy. Charles F Reynolds 3rd, Meryl A Butters, Oscar Lopez. et al. Arch Gen Psychiatry. 2011; 68 (1): 51-60).

It has also been common technical knowledge that smoking progresses and worsens the symptoms of novel coronavirus and that the cause thereof is nicotine (for example, Reference Literature 3: Tobacco smoking and COVID-19 infection. Richard N van Zyl-Smit, Guy Richards, Frank T Leone. Lancet Respir Med. July 2020; 8 (7): 664-665.; Reference Literature 4: Smoking Is Associated With COVID-19 Progression: A Meta-analysis. Roengrudee Patanavanich, Stanton A Glantz. Nicotine Tob Res. Aug. 24, 2020; 22 (9): 1653-1656.; Reference Literature 5: The Role of Smoking and Nicotine in the Transmission and Pathogenesis of COVID-19. Ali Ehsan Sifat, Saeideh Nozohouri, Heidi Villalba, Bhuvaneshwar Vaidya, Thomas J Abbruscato J Pharmacol Exp Ther. December 2020; 375 (3): 498-509.; Reference Literature 6: The Effect of Smoking on COVID-19 Symptom Severity: Systematic Review and Meta-Analysis Askin Gulsen 1, Burcu Arpinar Yigitbas 2, Berat Uslu 2, Daniel Dromann 1, Oguz Kilinc Pulm Med. September 8, 2020; 2020:7590207.).

In contrast, it can be said that the result that increasing the amount of acetylcholine in the brain by administering acetylcholine receptor agonists can improve novel coronavirus-related depressive symptoms exhibited in COVID-19 sequela model animals is completely opposite to the result expected from the conventional common technical knowledge as the effects of acetylcholine receptor agonists. Furthermore, the aforementioned result indicates that the depressive symptoms due to COVID-19 sequelae (that is, “novel coronavirus-related depressive symptoms”) arise from a different mechanism from depression caused by increased acetylcholine levels.

### Active Ingredient

The therapeutic agent in accordance with an aspect of the present invention contains an acetylcholine receptor agonist as an active ingredient. In the present specification, “acetylcholine receptor agonist” means both an indirect acetylcholine receptor agonist which increases the amount of acetylcholine in the brain through cholinesterase inhibitory action or the like and a direct acetylcholine receptor agonist which acts by directly binding to a receptor. In an aspect of the present invention, the acetylcholine receptor agonist may be a peripheral acetylcholine receptor agonist that acts through, for example, the parasympathetic nervous system or a central acetylcholine receptor agonist that acts on receptors in the brain. As discussed earlier, COVID-19 sequelae are symptoms related to the brain and nerve dysfunctions, and there is an advantage in being able to directly act on the sites where dysfunctions occur. Therefore, the acetylcholine receptor agonist is preferably a central acetylcholine receptor agonist that acts on intracerebral acetylcholine receptors. The “acetylcholine receptor agonist” is also referred to as a cholinergic agonist.

More specific examples of the intracerebral receptors on which the central acetylcholine receptor agonist acts include olfactory bulb, hippocampus, medial septum, and olfactory tubercle. However, because it is known that there are numerous sites where an acetylcholine receptor agonist acts, the intracerebral receptors are not limited to these examples.

The direct acetylcholine receptor agonist is preferably a substance that can cross the blood-brain barrier to reach the brain and have the effect of activating acetylcholine receptors. More specific examples of the direct acetylcholine receptor agonist include acetylcholine and precursors thereof, and agonists of acetylcholine receptors (muscarinic receptors or nicotinic receptors).

The indirect acetylcholine receptor agonist is preferably a substance that can cross the blood-brain barrier to reach the brain and have the effect of inhibiting acetylcholinesterase. More specific examples of the indirect acetylcholine receptor agonist include donepezil (2-[(1-benzyl-4-piperidyl) methyl]-5,6-dimethoxyindan-1-one hydrochloride); rivastigmine (2,6-dioxo-4-phenyl-piperidine-3-carbonitrile); metrifonate (O,O-dimethyl-2,2,2-trichloro-1-hydroxyethylphosphonate ester); tacrine (1,2,3,4-tetrahydro-9-aminoacridine); and galantamine (galantamine hydrobromide).

Among the acetylcholine receptor agonists exemplified, donepezil is already used as, for example, a therapeutic agent for Alzheimer's disease, and therefore drug repositioning is possible. Therefore, donepezil has the following advantages: (i) safety tests and pharmacokinetic tests on humans can be shortened or omitted, (ii) side effects are known, and therefore it is possible to select active ingredients that have minimal burden on patients, and (iii) the method for producing the active ingredient is already established, and therefore the pharmaceutical price can be kept low. Accordingly, in an aspect of the present invention, the acetylcholine receptor agonist is preferably donepezil.

It should be noted that the acetylcholine receptor agonist may be in the form of a “derivative” or a “pharmacologically acceptable salt”, provided that the acetylcholine receptor agonist has the effect of increasing the amount of acetylcholine in the brain. That is, in the present specification, the term “acetylcholine receptor agonist” is a concept that includes a “derivative” thereof and a “pharmacologically acceptable salt” thereof.

In the present specification, the term “derivative” of a specific compound refers to any of compounds derived from that specific compound as a result of substitution of a part of the molecule of the specific compound with some other functional group or some other atom. Examples of such other functional group include alkyl groups, alkoxy groups, alkylthio groups, aryl groups, aryloxy groups, arylthio groups, arylalkyl groups, arylalkoxy groups, arylalkylthio groups, arylalkenyl groups, arylalkyny groups, allyl group, amino groups, substituted amino groups, silyl groups, substituted silyl groups, silyloxy group, substituted silyloxy groups, arylsulfonyloxy groups, alkylsulfonyloxy groups, nitro group, and the like. Examples of such other atom include carbon atom, hydrogen atom, oxygen atom, nitrogen atom, sulfur atom, phosphorus atom, halogen atoms, and the like.

As derivatives, prodrugs that exhibit desired activity through, for example, hydrolysis, oxidization, or enzymatic reactions in vivo (under in vivo conditions), for example, can also be used.

In the present specification, “pharmaceutically acceptable salt” means a salt the administration of which as a pharmaceutical product to a subject is physiologically acceptable, and specific examples thereof are not limited. Examples of such a salt include alkali metal salts (such as a potassium salt), alkaline earth metal salts (such as a calcium salt and a magnesium salt), ammonium salts, organic basic salts (such as a trimethylamine salt, a triethylamine salt, a pyridine salt, a picoline salt, a dicyclohexylamine salt, and N,N′-dibenzylethylenediamine salt), organic acid salts (such as acetate, maleate, tartrate, methanesulfonate, benzenesulfonate, formate, toluene sulfonate, and trifluoroacetate), and inorganic acid salts (such as hydrochloride, hydrobromide, sulfate, and phosphate).

In an aspect of the present invention, the acetylcholine receptor agonist may activate or inhibit receptors other than acetylcholine receptors. However, from the viewpoint of suppressing unintended side effects and the like, the acetylcholine receptor agonist is preferably a compound that selectively (specifically) activates acetylcholine receptors.

### Other Components

The therapeutic agent in accordance with an aspect of the present invention may contain a component other than the aforementioned active ingredients (acetylcholine receptor agonists). The component other than the active ingredients need only be a pharmaceutically acceptable ingredient(s) and can be, for example, any of a buffer, a pH adjustor, a tonicity agent, an antiseptic agent, an antioxidant, a high molecular weight polymer, an excipient, and a solvent.

Examples of the buffer include phosphoric acid or phosphates, boric acid or borates, citric acid or citrates, acetic acid or acetates, carbonic acid or carbonates, tartaric acid or tartrates, ε-aminocaproic acid, trometamol, and the like. Examples of the phosphates include sodium phosphate, sodium dihydrogen phosphate, disodium hydrogen phosphate, potassium phosphate, potassium dihydrogen phosphate, dipotassium hydrogen phosphate, and the like. Examples of the borates include borax, sodium borate, potassium borate, and the like. Examples of the citrates include sodium citrate, disodium citrate, trisodium citrate, and the like. Examples of the acetates include sodium acetate, potassium acetate, and the like. Examples of the carbonates include sodium carbonate, sodium hydrogen carbonate, and the like. Examples of the tartrates include sodium tartrate, potassium tartrate, and the like.

Examples of the pH adjustor include hydrochloric acid, phosphoric acid, citric acid, acetic acid, sodium hydroxide, potassium hydroxide, and the like.

Examples of the tonicity agents include ionic tonicity agents (such as sodium chloride, potassium chloride, calcium chloride, and magnesium chloride) and nonionic tonicity agents (such as glycerin, propylene glycol, sorbitol, and mannitol).

Examples of the antiseptic agent include benzalkonium chloride, benzalkonium bromide, benzethonium chloride, sorbic acid, potassium sorbate, methyl parahydroxybenzoate, propyl parahydroxybenzoate, chlorobutanol, and the like.

Examples of the antioxidant include ascorbic acid, tocopherol, dibutylhydroxytoluene, butylated hydroxyanisole, sodium erythorbate, propyl gallate, sodium sulfite, and the like.

Examples of the high molecular weight polymer include methyl cellulose, ethyl cellulose, hydroxymethyl cellulose, hydroxyethyl cellulose, hydroxypropyl cellulose, hydroxyethyl methyl cellulose, hydroxypropyl methyl cellulose, carboxymethyl cellulose, carboxymethylcellulose sodium salt, hydroxypropyl methylcellulose acetate succinate, hydroxypropyl methylcellulose phthalate, carboxymethyl ethyl cellulose, cellulose acetate phthalate, polyvinylpyrrolidone, polyvinyl alcohol, carboxyvinyl polymer, polyethylene glycol, atelocollagen, and the like.

Examples of the excipient include lactose, saccharose, D-mannitol, xylitol, sorbitol, erythritol, starch, crystalline cellulose, and the like.

Examples of the solvent include water, physiological saline, alcohol, and the like.

The therapeutic agent in accordance with an aspect of the present invention may contain, as any of the aforementioned other components, a medicinal component having a desired effect (for example, of reducing a side effect) or may be used in combination with a drug having a desired effect.

### Amounts of Active Ingredients and Other Components Contained

The amount of the active ingredient in the therapeutic agent in accordance with an aspect of the present invention is not particularly limited. The amount of the active ingredient may be, for example, 0.001% by weight to 100% by weight, 0.01% by weight to 100% by weight, 0.1% by weight to 100% by weight, 0.1% by weight to 95% by weight, 0.1% by weight to 90% by weight, 0.1% by weight to 80% by weight, 0.1% by weight to 70% by weight, 0.1% by weight to 60% by weight, 0.1% by weight to 50% by weight, 0.1% by weight to 40% by weight, 0.1% by weight to 30% by weight, 0.1% by weight to 20% by weight, or 0.1% by weight to 10% by weight, with respect to the total weight of the therapeutic agent in accordance with an aspect of the present invention.

The amount of the components other than the active ingredient in the therapeutic agent in accordance with an aspect of the present invention is not particularly limited. The amount of the components other than the active ingredient may be, for example, 0% by weight to 99.999% by weight, 0% by weight to 99.99% by weight, 0% by weight to 99.9% by weight, 5% by weight to 99.9% by weight, 10% by weight to 99.9% by weight, 20% by weight to 99.9% by weight, 30% by weight to 99.9% by weight, 40% by weight to 99.9% by weight, 50% by weight to 99.9% by weight, 60% by weight to 99.9% by weight, 70% by weight to 99.9% by weight, 80% by weight to 99.9% by weight, or 90% by weight to 99.9% by weight, with respect to the total weight of the therapeutic agent in accordance with an aspect of the present invention.

### Administration Subject

Examples of the administration subject for the therapeutic agent in accordance with an aspect of the present invention include a subject suspected of being infected with the novel coronavirus or confirmed to be infected. Such an administration subject may be one who has not been diagnosed by a doctor for coronavirus sequelae.

The administration subject for the therapeutic agent in accordance with an aspect of the present invention is not particularly limited, and may be a human or a non-human mammal (such as a domestic animal, a pet, or a laboratory animal). Examples of the non-human mammal include monkeys, chimpanzees, cattle, pigs, sheep, goats, horses, dogs, cats, rabbits, mice, and rats.

### Administration Route

The therapeutic agent in accordance with an aspect of the present invention can be administered to the administration subject via any administration route. Examples of the administration route include oral administration, parenteral administration, transdermal administration, transmucosal administration, and intravenous administration. Therefore, the dosage form of the therapeutic agent in accordance with an aspect of the present invention may be, for example, an internal medicine, an external medicine, or an injection. The administration route of the therapeutic agent in accordance with an aspect of the present invention is preferably oral administration because the oral administration is easy and places less burden on the administration subject. Therefore, the dosage form of the therapeutic agent in accordance with an aspect of the present invention is preferably an internal medicine.

### Formulation and Prescription

The therapeutic agent in accordance with an aspect of the present invention can be formulated by a known method using, as raw materials, the acetylcholine receptor agonist which is an active ingredient and other components.

When the therapeutic agent in accordance with an aspect of the present invention is administered to a subject, there is no limitation on the amount of the administration to the subject, provided that a desired effect is obtained. For example, the therapeutic agent in accordance with an aspect of the present invention may be administered so that the dosage of the acetylcholine receptor agonist, which is an active ingredient, is 0.1 mg/kg body weight to 1000.0 mg/kg body weight, 0.1 mg/kg body weight to 500.0 mg/kg body weight, 1.0 mg/kg body weight to 500.0 mg/kg body weight, 1.0 mg/kg body weight to 300.0 mg/kg body weight, 1.0 mg/kg body weight to 100.0 mg/kg body weight, 1.0 mg/kg body weight to 50.0 mg/kg body weight, 1.0 mg/kg body weight to 10.0 mg/kg body weight, 1.0 mg/kg body weight to 10.0 mg/kg body weight, or 1.0 mg/kg body weight to 5.0 mg/kg body weight.

When the therapeutic agent in accordance with an aspect of the present invention is administered to a subject, there is no limitation on the intervals of the administration to the subject, provided that a desired effect is obtained. The dosing interval may be, for example, once for every hour, once for 1 hour to 6 hours, once for 6 hours to 12 hours, once for 12 hours to 1 day, once for 1 day to 3 days, once for 1 day to 5 days, once for 1 day to 7 days, once for 7 days to 14 days, once for 14 days to 21 days, once for every month, once for 2 months, once for 3 months, once for 4 months, once for 5 months, once for 6 months, or once for every year. In addition, although the dosing interval may be constant, the therapeutic agent may be administered (dosed) whenever the symptoms of COVID-19 sequelae become pronounced.

The therapeutic agent in accordance with an aspect of the present invention may be administered, for example, once per day at a dosage of the acetylcholine receptor agonist, which is an active ingredient, ranging from 1.0 mg/kg body weight to 5.0 mg/kg body weight.

### 2. Drug Screening Method

### Features

A COVID-19 sequela therapeutic agent screening method in accordance with an aspect of the present invention uses COVID-19 sequela model animals, which are obtained by expressing SARS-COV-2 S1 protein in non-human mammals, for screening the COVID-19 sequela therapeutic agent that has an effect of treating or preventing COVID-19 sequelae, and includes: an administration step of administering a test substance into a COVID-19 sequela model animal; and an evaluation step of evaluating a change in the novel coronavirus-related symptoms of the COVID-19 sequela model animal before and after the administration of the test substance. This allows for screening of the therapeutic agent for COVID-19 sequelae, and thus leads to the development of new therapeutic agent for COVID-19 sequelae. Therefore, it is possible to contribute to “Good Health and Well-Being”, which is Goal 3 of the sustainable development goals (SDGs). Hereinafter, for convenience of explanation, the COVID-19 sequela therapeutic agent screening method in accordance with an aspect of the present invention may be simply referred to as “drug screening method”.

### COVID-19 Sequela Model Animal

The COVID-19 sequela model animals used in the COVID-19 sequela therapeutic agent screening method in accordance with an aspect of the present invention are obtained by expressing SARS-COV-2 S1 protein in non-human mammals.

In a COVID-19 sequela model animal, SARS-COV-2 S1 protein may be expressed transiently or continuously. Transgenic animals may be used. Since it is possible to examine the symptoms at a point in time when the expression has ended or diminished, it is preferable that the expression of the SARS-COV-2 S1 protein is transient and that, by the time of the use in the administration step of the present screening method, the expression of the SARS-COV-2 S1 protein in the COVID-19 sequela model animal has ended or diminished.

In addition, since it is possible to efficiently induce COVID-19 sequelae, the COVID-19 sequela model animal is preferably a model animal obtained by expressing the SARS-CoV-2 S1 protein in at least one of a nasal cavity and a part around the nasal cavity of a non-human mammal. The COVID-19 sequela model animal may have inflammation induced before the step of administering the test substance.

The method for expressing the SARS-COV-2 S1 protein in a non-human mammal is not particularly limited. For example, a COVID-19 sequela model animal can be produced by using the COVID-19 sequela model animal production method discussed later to express the SARS-COV-2 S1 protein in a non-human mammal.

### Administration Step

The administration step is a step of administering a test substance into a COVID-19 sequela model animal. The amount and method of administering the test substance are not particularly limited. The amount and method can be set as appropriate according to the active ingredient concentration in the test substance, the dosage form of the test substance, and the like.

### Evaluation Step

The evaluation step is a step of evaluating the changes in novel coronavirus-related symptoms of a COVID-19 sequela model animal before and after the administration of a test substance.

In the evaluation step, the method for evaluating the changes in novel coronavirus-related symptoms of a COVID-19 sequela model animal is not particularly limited. For example, in the evaluation step, the changes in novel coronavirus-related symptoms of the COVID-19 sequela model animal can be evaluated by quantifying the changes in the activity level of the COVID-19 sequela model animal.

As a method for quantifying the changes in the activity level of the COVID-19 sequela model animal, behavioral experiments such as the 10% weight-loaded forced swimming test and the tail suspension test, which are discussed later in Examples, can be conducted before and after the administration of the test substance so as to quantify the changes in the activity level of the COVID-19 sequela model animal before and after the administration of the test substance.

In addition, for example, in the evaluation step, the changes in novel coronavirus-related symptoms of the COVID-19 sequela model animal may be confirmed by quantifying the changes in the expression level of inflammation markers in the brain of the COVID-19 sequela model animal. Examples of the inflammation markers include interleukin-6 (IL-6), tumor necrosis factor (TNFα), chemokine CC motif ligand 2 (CCL2).

When the changes in the expression levels of inflammation markers in the brain of the COVID-19 sequela model animal are to be quantified, it is not possible to quantify the changes in the expression levels of inflammation markers in the brain of the same COVID-19 sequela model animal before and after the administration of the test substance. Therefore, by comparing the measurement of the expression levels of the aforementioned inflammation markers between the test substance administration group and the placebo administration group, it is possible to quantify the changes in the expression levels of inflammation markers in the brain of the COVID-19 sequela model animal before and after the administration of the test substance.

In the evaluation step, based on the results of the evaluation of the changes in novel coronavirus-related symptoms, the therapeutic effect or preventive effect of the test substance on COVID-19 sequelae can be evaluated. For example, the test substance can be evaluated as having the activity to treat or prevent COVID-19 sequelae if in the evaluation step, the changes in novel coronavirus-related symptoms of the COVID-19 sequela model animal are evaluated by quantifying the changes in the activity level of the COVID-19 sequela model animal and it is as a result found that novel coronavirus-related fatigue or depressive symptoms are improved before and after the administration of the test substance.

In addition, the test substance can be evaluated as having the activity to treat or prevent COVID-19 sequelae if in the evaluation step, for example, the changes in novel coronavirus-related symptoms of the COVID-19 sequela model animal are evaluated by quantifying the changes in the expression levels of inflammation markers in the brain of the COVID-19 sequela model animal and it is as a result found that the expression levels of the inflammation markers in the brain are significantly decreased before and after the administration of the test substance.

The screening by quantifying the changes in the activity level of the COVID-19 sequela model animal and the screening by quantifying the changes in the expression levels of inflammation markers in the brain of the COVID-19 sequela model animal can be used in combination as appropriate.

In the drug screening method in accordance with an aspect of the present invention, it is also possible to narrow down effective test substances. For example, Example 6 discussed later demonstrated that there is deficiency of acetylcholine in the brain having COVID-19 sequela. Thus, acetylcholine receptor agonists can be set as candidate drugs with high priority.

Furthermore, Example 10 discussed later demonstrated that, among the acetylcholine receptor agonists, the administration of PNU282987, which is an α7 nicotinic receptor agonist that does not pass through the blood-brain barrier, into the brain ventricles has an effect of suppressing brain inflammation. This result suggests that the priority of α7 nicotinic receptor agonists should be increased in future screenings. This indicates that even drugs that do not pass through the blood-brain barrier and thus are not realistic therapeutic agent candidates can be utilized, by a method such as intracerebroventricular administration, for suggesting, for example, a drug screening method.

Although it was known that α7 nicotinic receptor agonists have immunosuppressive functions, the mechanism thereof was not clear. Example 11 discussed later suggests that the mechanism involves the increased expression of the immunosuppressive molecule ZFP36 induced by α7 nicotinic receptor agonists. This result suggests that by elucidating the mechanisms of therapeutic effects of drugs during the screening step, it is possible to develop superior effect determination methods and to identify the target molecules of the drugs.

### 3. Model Animal Production Method

### Features

The COVID-19 sequela model animal production method in accordance with an aspect of the present invention includes an expression step of expressing SARS-COV-2 S1 protein in a non-human mammal with use of a SARS-COV-2 S1 protein expression vector. This makes it possible to produce a COVID-19 sequela model animal by expressing the SARS-COV-2 S1 protein in a non-human mammal. The COVID-19 sequela model animal can be used as an experimental animal in the development of a method for treating or preventing COVID-19 sequelae, particularly in the development of drug such as a therapeutic agent and a preventive agent. In addition, the COVID-19 sequela model animal can be used as an experimental animal in research on the causes of the onset of COVID-19 sequelae. Therefore, it is possible to contribute to “Good Health and Well-Being”, which is Goal 3 of the sustainable development goals (SDGs).

### Types of Model Animals

The types of COVID-19 sequela model animals produced by the COVID-19 sequela model animal production method in accordance with an aspect of the present invention are not limited to any particular ones, provided that the COVID-19 sequela model animals are non-human mammals (mammals other than humans) that can be used as experimental animals. Therefore, the type of non-human mammal targeted in the expression step is not particularly limited and can be selected as appropriate according to the intended use of the model animal being produced. Examples of the non-human mammal that can be targeted in the expression step include mice, rats, guinea pigs, dogs, rabbits, monkeys, and chimpanzees.

Regarding animals infected with the novel coronavirus, it is known that large animals such as monkeys are effective. However, in the development of treatment or prevention methods, particularly in the development of drugs such as therapeutic agents and preventive agents, it is considered necessary to use animal models with small animals such as mice. For these reasons, the COVID-19 sequela model animal is preferably a small animal model such as a mouse model.

### Expression Step

The expression step is a step of expressing the SARS-CoV-2 S1 protein in a non-human mammal with use of a SARS-COV-2 S1 protein expression vector. By expressing the SARS-COV-2 S1 protein in a non-human mammal with use of the SARS-COV-2 S1 protein expression vector, it is possible to induce COVID-19 sequela in the non-human mammal.

In viral infections, it is generally known that an immune response produces inflammatory cytokines, which causes fatigue and depressive symptoms during the acute phase of the infection. However, it is known that COVID-19 sequelae exhibit fatigue and depressive symptoms more severe than those observed with other viruses, and that these symptoms persist as sequelae. Thus, the novel coronavirus presumably possesses a protein with strong activity that causes neurological damage during infection. Therefore, the inventors of the present invention conducted diligent studies to identify such a protein and, as a result, discovered for the first time that the S1 region of the Spike protein (1273 amino acids, GenBank accession number YP 009724390) of the SARS-COV-2 virus is the causative protein of COVID-19 sequelae.

Handling model animals prepared by infection with the infectious SARS-COV-2 virus itself requires advanced containment facilities such as P3 facility. In contrast, the COVID-19 sequela model animal production method in accordance with an aspect of the present invention involves expressing the SARS-COV-2 S1 protein, which is the causative protein of COVID-19 sequelae, in a non-human mammal with use of a SARS-Cov-2 S1 protein expression vector instead of the SARS-COV-2 virus itself. Thus, model animals produced by the COVID-19 sequela model animal production method in accordance with an aspect of the present invention can be used in an ordinary experimental environment, and are excellent in terms of handleability.

In model animals prepared by infection with the infectious SARS-COV-2 virus itself, the pathogenicity of acute infection and the pathogenicity of sequelae may not occur to the same extent as in humans. Therefore, it is possible that the survival rate of the model animals due to acute symptoms is low, or that sufficient sequelae do not develop as models. Thus, the COVID-19 sequela model animal production method in accordance with an aspect of the present invention are excellent in that the model animals can be efficiently and reliably produced.

In the present specification, the phrase “expressing the SARS-COV-2 S1 protein in a non-human mammal with use of a SARS-COV-2 S1 protein expression vector” means expressing the SARS-COV-2 S1 protein within the body of the target non-human mammal with use of the SARS-COV-2 S1 protein expression vector.

The site within the body of the target non-human mammal where the SARS-COV-2 S1 protein is to be expressed is not particularly limited. However, to be able to efficiently induce COVID-19 sequelae, the SARS-COV-2 S1 protein is preferably expressed in at least one of a nasal cavity and a part around the nasal cavity of the non-human mammal in the expression step. To express the SARS-Cov-2 S1 protein in at least one of a nasal cavity and a part around the nasal cavity of the non-human mammal, the expression step may, for example, involve administering the SARS-COV-2 S1 protein expression vector to the nasal cavity of the non-human mammal.

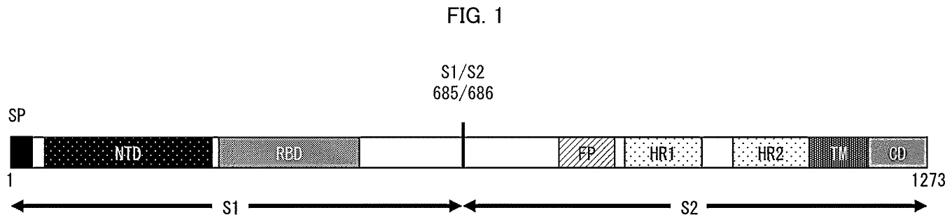

The structure of the SARS-COV-2 Spike protein is illustrated in FIG. 1. The S1 region is a region that has an amino acid sequence constituted by the 1st to the 685th amino acids in the amino acid sequence of the SARS-COV-2 Spike protein. The S1 region includes the signal peptide sequence (SP), the N-terminal domain (NTD), and the receptor-binding domain (RBD). In the present specification, a polypeptide containing the S1 region of the Spike protein of the SARS-COV-2 virus is referred to as “SARS-COV-2 S1 protein” or simply “S1 protein”.

The SARS-COV-2 S1 protein may be, for example, one of the following polypeptides (a) and (b):
(a) a polypeptide having the amino acid sequence (SEQ ID NO: 1) constituted by the 1st to the 685th amino acids in the amino acid sequence shown in GeneBank accession number YP_009724390;
(b) a polypeptide constituted by an amino acid sequence having a sequence identity of 80% or more to the amino acid sequence (SEQ ID NO: 1) constituted by the 1st to the 685th amino acids in the amino acid sequence shown in GeneBank accession number YP_009724390 and having the activity of raising an intracellular calcium concentration when introduced into a cell.

The SARS-COV-2 S1 protein is a polypeptide consisting of 685 amino acids with a molecular weight of approximately 76.7 kDa.

As in the polypeptide (b) above, the SARS-COV-2 S1 protein may be a polypeptide constituted by an amino acid sequence having a sequence identity of 80% or more (85% or more, 90% or more, 95% or more, 98% or more, 99% or more) to the amino acid sequence (SEQ ID NO: 1) constituted by the 1st to the 685th amino acids in the amino acid sequence shown in GeneBank accession number YP_009724390 and having the activity of raising an intracellular calcium concentration when introduced into a cell. In the present specification, the percentage of amino acid sequence identity is calculated using genetic information processing software GENETYX Ver. 7 (manufactured by GENETYX CORPORATION).

The polypeptide (b) may be a polypeptide constituted by an amino acid sequence in which 100 or less amino acids are substituted, deleted, inserted, and/or added in the amino acid sequence (SEQ ID NO: 1) constituted by the 1st to the 685th amino acids in the amino acid sequence shown in GeneBank accession number YP_009724390, and having the activity of raising an intracellular calcium concentration when introduced into a cell. In the present specification, the phrase “100 or less amino acids are substituted, deleted, inserted, and/or added” means that 100 or less (90 or less, 80 or less, 70 or less, 60 or less, 50 or less, 40 or less, 30 or less, 20 or less, 15 or less, 10 or less, 7 or less, 5 or less, or 2 or less) amino acids are substituted, deleted, inserted, and/or added by a known method for preparing mutant peptides, such as site-directed mutagenesis. Thus, the polypeptide (b) can be said to be a mutant of the polypeptide (a). It should be noted that the term “mutation” as used here primarily refers to a mutation artificially introduced by a known method for preparing mutant proteins. However, the term “mutation” may also include similar mutant proteins that are naturally-occurring and have been isolated and purified.

Mutant strains of the SARS-COV-2 virus have been reported so far and are known to have mutations in the spike protein. The main mutations in the SARS-COV-2 S1 protein in the reported mutant strains are as follows. The polypeptide (b) preferably has these mutations.
SARS-COV-2 B.1.1.7 lineage (so-called “Alpha variant”): deletion69-70, deletion 144-145, N501Y, A570D, D614G and P681H
SARS-COV-2 B.1.351 lineage (so-called “Beta variant”): D80A, D215G, Deletion241-243, K417N, E484K, N501Y, and D614G.
SARS-COV-2 P.1 lineage (so-called “Gamma variant”): L18F, T20N, P26S, D138Y, R190S, K417T, E484K, N501Y, D614G, and H655Y
SARS-COV-2 B.1.617.2 lineage (so-called “Delta variant”): T19R, G142D, E156G, deletion 157-158, L452R, T487K, E484Q, D614G, and P681R
SARS-COV-2 B.1.1.529 lineage (so-called “Omicron variant”): G142D, G339D, S371L, S373P, S375F, K417N, N440K, G446S, S477N, T478K, E484A, Q493R, G496S, Q498R, N501Y, Y505H, D614G, H655Y, N679K, and P681H

The fact that the mutant protein of the SARS-COV-2 S1 protein has the activity of raising an intracellular calcium concentration can be confirmed by expressing the mutant proteins in any cultured cells and measuring the intracellular calcium concentrations. An intracellular calcium concentration can be measured by the method discussed later in Examples. If the calcium concentration in cells expressing the mutant protein is significantly higher in comparison with that in the parent cells, it can be determined that the mutant protein has the activity of raising an intracellular calcium concentration.

The SARS-COV-2 S1 protein expression vector is an expression vector that is obtained by introducing a polynucleotide encoding the SARS-COV-2 S1 protein into a known plasmid vector or viral vector.

The plasmid vector or viral vector can be selected as appropriate from known vectors commonly used in genetic recombination as long as the vector can express the SARS-CoV-2 S1 protein within the body of a non-human mammal, and the type is not particularly limited. A viral vector can be suitably used, due to high infection efficiency and ease of introduction into non-human mammals. Examples of a particularly suitable viral vector include adenovirus vectors, which are commonly used for purposes such as known gene therapy and viral vaccines.

The type of promoter for expressing the SARS-COV-2 S1 protein is not limited to any particular one, provided that the promoter can express mRNA in a cell that SARS-COV-2 infects. A promoter that can produce the SARS-COV-2 S1 protein at a level substantially equal to that during SARS-COV-2 infection is particular suitable for use.

The polynucleotide encoding the SARS-COV-2 S1 protein may be, for example, a polynucleotide encoding the aforementioned polypeptide (b) or (a).

As an example, the full genome sequence of SARS-COV-2 S1, which includes the nucleotide sequence of the polynucleotide encoding the polypeptide (a), is published under Gen Bank accession number MN908947. The polynucleotide encoding the polypeptide (a) has a nucleotide sequence (SEQ ID NO: 2) constituted by the 21563rd to the 23617th nucleotides in the nucleotide sequence shown in GeneBank accession number MN908947. The polynucleotide encoding the polypeptide (a) has a size of 2055 base pairs (approximately 2 kbp).

The method for obtaining the polynucleotide encoding the SARS-COV-2 S1 protein is not particularly limited. Examples of such a method include those using amplification techniques. For example, primers are prepared from the 5′- and 3′-side sequences (or sequences complementary thereto) of cDNA of the gene encoding the SARS-COV-2 Spike protein, and PCR or the like is performed using these primers and using genomic DNA (or cDNA) or the like as a template, and thereby a DNA region between the primers is amplified. In this way, a large amount of DNA fragments containing the polynucleotide encoding the SARS-COV-2 S1 protein can be gene sequence information, a obtained. Based on polynucleotide having the nucleotide sequence of the polynucleotide encoding the SARS-COV-2 S1 protein may be synthesized using a known chemical synthesis method. The nucleotide sequence may be a sequence that is optimized for the codon usage frequency of the animal used.

In the expression step, the expression level of SARS-CoV-2 S1 protein need only be adjusted in view of the type and body weight of the target non-human mammal so that a sufficient amount of SARS-COV-2 S1 protein is expressed to induce COVID-19 sequelae.

In the expression step of the COVID-19 sequela model animal production method in accordance with an aspect of the present invention, the SARS-COV-2 S1 protein may be transiently or continuously expressed in a non-human mammal. For research on COVID-19 sequelae using the COVID-19 sequela model animal, it is desirable to be able to examine the symptoms at a point in time when the expression of the SARS-COV-2 S1 protein has ended or diminished. Therefore, in the expression step, it is preferable to transiently express the SARS-COV-2 S1 protein in the non-human mammal.

### Inflammation Inducing Step

It is preferable that the COVID-19 sequela model animal production method in accordance with an aspect of the present invention further includes an inflammation inducing step of inducing inflammation in the non-human mammal. In COVID-19 sequelae, the SARS-COV-2 S1 protein is expressed in an inflammatory state caused by SARS-COV-2 viral infection. Therefore, the COVID-19 sequela model animal production method in accordance with an aspect of the present invention further includes the inflammation inducing step. This makes it possible to prepare model animals that take into account the actual conditions of SARS-COV-2 viral infection.

The method for inducing inflammation in a non-human mammal is not particularly limited; however, from the viewpoint of handling the model animals prepared, it is preferable to induce inflammation using agents rather than viral infection. For example, inflammation in a non-human mammal can be induced by intraperitoneal administration of lipopolysaccharide (LPS) derived from Gram-negative bacteria such as E. coli O111. It is known that LPS has biological activity that activates macrophages.

The inflammation inducing step is performed after the expression step. By performing the inflammation inducing step after the expression step, it is possible to observe the effect of inducing inflammation in model animals exhibiting COVID-19 sequelae. The inflammation inducing step may be performed before the expression step.

### Evaluation Step

The COVID-19 sequela model animal production method in accordance with an aspect of the present invention may further include an evaluation step of evaluating the extent of COVID-19 sequelae symptoms in the non-human mammal after the expression step.

The representative symptoms of COVID-19 sequelae are fatigue and depressive symptoms. Thus, by behavioral experiments and the quantification thereof to evaluate the extent of fatigue or depressive symptoms, it is possible to evaluate the extent of COVID-19 sequelae symptoms in the non-human mammal after the expression step.

The extent of fatigue can be evaluated by conducting the 10% weight-loaded forced swimming test discussed later in Examples. The extent of depressive symptoms can be evaluated by conducting the tail suspension test discussed later in Examples.

By performing the evaluation step, it is possible to confirm that the model animals produced by the COVID-19 sequela model animal production method in accordance with an aspect of the present invention are indeed exhibiting symptoms of COVID-19 sequelae. According to the results of the evaluation step, it is possible to adjust as appropriate, for example, the amount of SARS-COV-2 S1 protein expressed in the expression step, the type of expression vector in the expression step, and the extent of inflammation induced in the inflammation inducing step.

### 4. Kit for Preparing COVID-19 Sequela Model Animal

A COVID-19 sequela model animal preparation kit in accordance with an aspect of the present invention (hereinafter simply referred to as “kit”) includes an expression vector that is capable of expressing the SARS-COV-2 S1 protein within a cell of a non-human mammal. The kit in accordance with an aspect of the present invention can be suitably used in the COVID-19 sequela model animal production method in accordance with an aspect of the present invention.

In an aspect of the present invention, the term “kit” refers to a package including a container (such as a bottle, a plate, a tube, or a dish) that encloses certain materials. The kit in accordance with an aspect of the present invention may contain each of the materials independently, or may contain a mixture of a plurality of materials (for example, in the form of a composition). The kit preferably includes an instruction manual for using each of the materials.

The kit in accordance with an aspect of the present invention need only include materials for performing the COVID-19 sequela model animal production method in accordance with an aspect of the present invention. The specific configurations, materials, equipment and the like of the kit, other than the expression vector capable of expressing the SARS-COV-2 S1 protein in a cell of a non-human mammal, are not particularly limited.

In addition to the expression vector capable of expressing the SARS-COV-2 S1 protein in a cell of a non-human mammal, the kit in accordance with an aspect of the present invention may further include lipopolysaccharide (LPS) for use in the inflammation inducing step.

### 5. COVID-19 Sequela Model Animal

A COVID-19 sequela model animal produced by the COVID-19 sequela model animal production method in accordance with an aspect of the present invention is also encompassed in the scope of the present invention. Since the COVID-19 sequela model animal in accordance with an aspect of the present invention is produced by the COVID-19 sequela model animal production method in accordance with an aspect of the present invention, the COVID-19 sequela model animal does not require advanced containment facilities such as P3 facility and can be used in an ordinary experimental environment. This makes the COVID-19 sequela model animal excellent in terms of handling.

### Additional Remarks

As has been discussed, an aspect of the present invention is as follows:
<1> A therapeutic agent for a sequela of a novel coronavirus infection, said therapeutic agent containing an acetylcholine receptor agonist as an active ingredient.
<2> The therapeutic agent according to <1>, in which the acetylcholine receptor agonist is a central acetylcholine receptor agonist that acts on an intracerebral acetylcholine receptor.
<3> The therapeutic agent according to <1> or <2>, in which the acetylcholine receptor agonist is donepezil.
<4> The therapeutic agent according to any one of <1> through <3>, in which the sequela of the novel coronavirus infection is novel coronavirus-related fatigue.
<5> The therapeutic agent according to any one of <1> through <3>, in which the sequela of the novel coronavirus infection is a novel coronavirus-related depressive symptom.
<6> The therapeutic agent according to any one of <1> through <3>, in which the sequela of the novel coronavirus infection is a novel coronavirus-related olfactory dysfunction.
<7> The therapeutic agent according to any one of <1> through <3>, in which the sequela of the novel coronavirus infection is a novel coronavirus-related memory impairment.
<8> A method for screening a therapeutic agent for a sequela of a novel coronavirus infection, said method including: administering a test substance into a novel coronavirus infection sequela model animal obtained by expressing SARS-CoV-2 S1 protein in a non-human mammal; and
   evaluating a change in a novel coronavirus-related symptom of the model animal before and after administration of the test substance.
<9> The method according to <8>, in which the model animal is a model animal obtained by expressing SARS-COV-2 S1 protein in at least one of a nasal cavity and a part around the nasal cavity of the non-human mammal.
<10> The method according to <8> or <9>, in which the model animal is a model animal produced by a novel coronavirus infection sequela model animal production method including expressing SARS-COV-2 S1 protein in the non-human mammal with use of a SARS-COV-2 S1 protein expression vector.
<11> The method according to <10>, in which the novel coronavirus infection sequela model animal production method further includes inducing inflammation in the non-human mammal.
<12> A method for producing a novel coronavirus infection sequela model animal, including expressing SARS-COV-2 S1 protein in a non-human mammal with use of a SARS-COV-2 S1 protein expression vector.
<13> The method according to <12>, in which, in the expressing, the SARS-COV-2 S1 protein is expressed in at least one of a nasal cavity and a part around the nasal cavity of the non-human mammal.
<14> The method according to <12> or <13>, further including inducing inflammation in the non-human mammal.
<15> The method according to any one of <12> through <14>, in which the SARS-COV-2 S1 protein is one of the following polypeptides (a) and (b):
   (a) a polypeptide having the amino acid sequence (SEQ ID NO: 1) constituted by the 1st to the 685th amino acids in the amino acid sequence shown in GeneBank accession number YP_009724390;
   (b) a polypeptide constituted by an amino acid sequence having a sequence identity of 80% or more to the amino acid sequence (SEQ ID NO: 1) constituted by the 1st to the 685th amino acids in the amino acid sequence shown in GeneBank accession number YP_009724390 and having the activity of raising an intracellular calcium concentration when introduced into a cell.

### 6. Method for Treating or Preventing COVID-19 Sequelae

A method for treating or preventing COVID-19 sequelae with use of the COVID-19 sequela therapeutic agent in accordance with an aspect of the present invention is also encompassed in the scope of the present invention.

That is, the COVID-19 sequela treating or preventing method in accordance with an aspect of the present invention is as follows:
<16> A method for treating or preventing a COVID-19 sequela, the method including the step of administering, into a subject (such as a human or a non-human animal), a COVID-19 sequela therapeutic agent that contains an acetylcholine receptor agonist as an active ingredient.
<17> The method according to <16>, in which the acetylcholine receptor agonist is a central acetylcholine receptor agonist that acts on an intracerebral acetylcholine receptor.
<18> The method according to <16> or <17>, in which the acetylcholine receptor agonist is donepezil.
<19> The method according to any one of <16> through <18>, in which the COVID-19 sequela is novel coronavirus-related fatigue.
<20> The method according to any one of <16> through <18>, in which the COVID-19 sequela is a novel coronavirus-related depressive symptom.
<21> The method according to any one of <16> through <18>, in which the COVID-19 sequela is a novel coronavirus-related olfactory dysfunction.
<22> The method according to any one of <16> through <18>, in which the COVID-19 sequela is a novel coronavirus-related memory impairment.

The administration subject, the administration route, the formulation, and the prescription, for example, of the therapeutic agent for COVID-19 sequelae are as explained for the therapeutic agent in accordance with an aspect of the present invention, and therefore will not be repeated here. The therapeutic agent for COVID-19 sequelae is preferably administered orally, because oral administration is easy and places less burden on the administration subject.

### 7. Other Remarks

The use of an acetylcholine receptor agonist for producing the COVID-19 sequela therapeutic agent in accordance with an aspect of the present invention is also encompassed in the scope of the present invention.

That is, the use in accordance with an aspect of the present invention is as follows:
<23> Use of an acetylcholine receptor agonist for producing a therapeutic agent for a COVID-19 sequela.
<24> The use according to <23>, in which the acetylcholine receptor agonist is a central acetylcholine receptor agonist that acts on an intracerebral acetylcholine receptor.
<25> The use according to <23> or <24>, in which the acetylcholine receptor agonist is donepezil.
<26> The use according to any one of <23> through <25>, in which the COVID-19 sequela is novel coronavirus-related fatigue.
<27> The use according to any one of <23> through <25>, in which the COVID-19 sequela is a novel coronavirus-related depressive symptom.
<28> The use according to any one of <23> through <25>, in which the COVID-19 sequela is a novel coronavirus-related olfactory dysfunction.
<29> The use according to any one of <23> through <25>, in which the COVID-19 sequela is a novel coronavirus-related memory impairment.

The present invention is not limited to the embodiments, but can be altered by a skilled person in the art within the scope of the claims. The present invention also encompasses, in its technical scope, any embodiment derived by combining technical means disclosed in differing embodiments.

### EXAMPLES

### Example 1

The neurotoxicity of the SARS-COV-2 S1 protein was examined using the increase in intracellular calcium as an indicator.

### Method

### Construction of Expression Vector for Mammalian Cells

A DNA fragment (2055 base pairs) encoding the S1 region (685 amino acids) of the SARS-COV-2 Spike protein (1273 amino acids, Gen Bank accession number YP_009724390) was incorporated into the mammalian cell expression vector pFlag-CMV-5a to construct the S1 expression vector plasmid (S1/pFlag). The S1 region of the SARS-COV-2 Spike protein (hereinafter simply referred to as “S1 protein”) is a polypeptide having the amino acid sequence (SEQ ID NO: 1) constituted by the 1st to the 685th amino acids in the amino acid sequence shown in GeneBank accession number YP_009724390. The nucleotide sequence of the DNA fragment encoding the S1 protein is published under GenBank accession number MN908947.

Furthermore, the Adenovirus Dual Expression Kit (TaKaRa) was used to construct the S1-expressing adenovirus vector. A DNA fragment encoding the S1 region was incorporated into the cosmid vector pAxCAwtit2, and an adenovirus vector (S1/Adv) was constructed in which S1 is expressed under control of the CAG promoter.

### Measurement of Intracellular Calcium

To express the S1 protein in mouse and human cells, the mouse fibroblast cell line 3T3 derived from skin and the human alveolar basal epithelial adenocarcinoma cell line A549 were used. The S1/pFlag was introduced into these cells using the CalPhos Mammalian Transfection Kit (TakaRa) to transiently express the S1 protein. As a control, an empty vector plasmid pFlag-CMV-5a was used.

In the case of the adenovirus vector, the S1/Adv was directly infected into these cells to transiently express the S1 protein. As a control, an empty adenovirus vector was used.

To measure the intracellular calcium concentration in cells expressing the S1 protein using the aforementioned method, Calcium Kit II—Fluo 4 (DOJINDO) was used to bind calcium ions and the fluorescent probe, and the fluorescence intensity of each cell was measured using ArrayScan XT (ThermoFisher).

### Results

As illustrated in FIG. 1, the S1 protein used in the construction of the expression vector is a protein having the amino acid sequence (SEQ ID NO: 1) constituted by the 1st to the 685th amino acids in the amino acid sequence of the SARS-COV-2 Spike protein shown in GeneBank accession number YP_009724390.

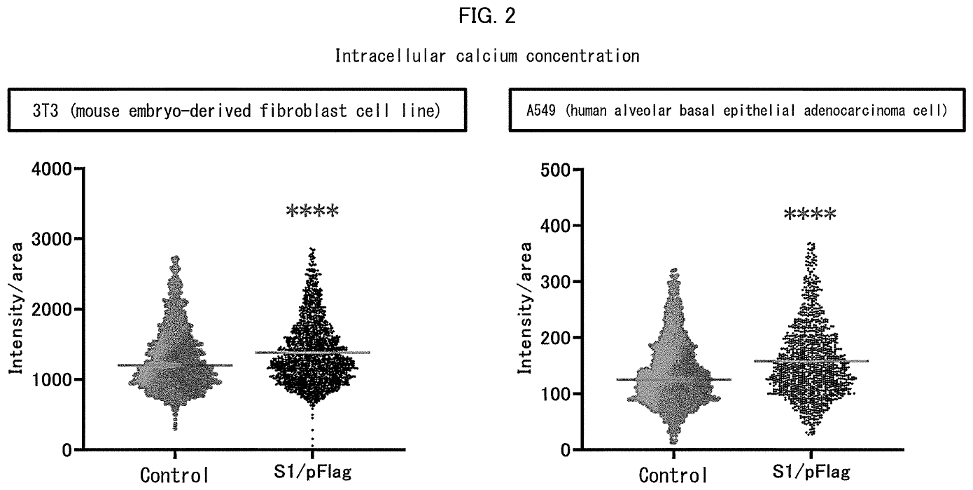

The 3T3 cells and A549 cells were transfected with the S1 protein expression plasmid or the control plasmid, and the intracellular calcium concentration was measured. The results are shown in FIG. 2. “Intensity/area” on the vertical axis of the graph in FIG. 2 indicates the fluorescence intensity per unit area. “Control” on the horizontal axis of the graph in FIG. 2 indicates the cells transfected with the control plasmid, and “S1/pFlag” indicates the cells transfected with the S1 protein expression plasmid. The horizontal bars shown in FIG. 2 indicate the median values. ****: P<0.0001.

As illustrated in FIG. 2, the intracellular calcium concentration increased with the expression of the S1 protein in both cell types.

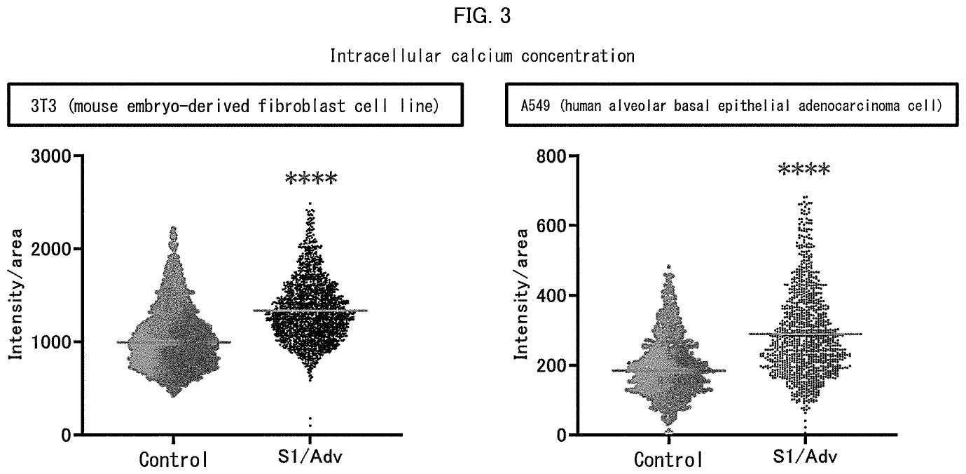

Furthermore, the intracellular calcium concentrations in 3T3 cells and A549 cells infected with the S1 protein-expressing adenovirus or the control adenovirus were measured. The results are shown in FIG. 3. “Intensity/area” on the vertical axis of the graph in FIG. 3 indicates the fluorescence intensity per unit area. “Control” on the horizontal axis of the graph in FIG. 3 indicates the cells infected with the control adenovirus, and “S1/Adv” indicates the cells infected with the S1 protein-expressing adenovirus. The horizontal bars shown in FIG. 3 indicate the median values. ****: P<0.0001.

As illustrated in FIG. 3, the intracellular calcium concentration increased with the expression of the S1 protein in both cell types (FIG. 3).

Therefore, it was revealed that the intracellular calcium concentration increases with the expression of the S1 protein in both mouse and human cells. This suggests a possibility that when the S1 protein is expressed in nerve cells, the intracellular calcium concentration increases and induces nerve cell death.

### Example 2

### Fatigue Manifestation in S1 Protein-Expressing Mice (S1 Mice)

### Method

### Preparation of S1 Protein-Expressing Mice (S1 Mice)

C57BL/6 mice of 8 weeks old to 9 weeks old were anesthetized using isoflurane. 25 μL of 1×10⁹ ifu/mL S1/Adv solution was administered into the nasal cavities and inhaled through natural breathing (expression step) to prepare S1 mice (COVID-19 sequela model animals). The mice were then returned to the home cages and kept for one week. As a control, mice that were administered, through nasal administration, with empty adenovirus vector (vector/Adv) which did not express anything were used (control mice).

### Fatigue Behavioral Test

On the 6th day after nasal administration of S1/Adv or vector/Adv, a 10% weight-loaded forced swimming test was conducted as a fatigue behavioral test. As the method, the body weights of the S1 mice and the control mice were measured in the morning of the test day, and weights corresponding to approximately 10% of the body weights of the mice were prepared. The weights were fixed to the tails of the S1 mice or control mice, and the mice were placed in a water tank for the forced swimming test. The period of time until the nose of the mouse was submerged below the water surface for 10 seconds was measured.

### Results

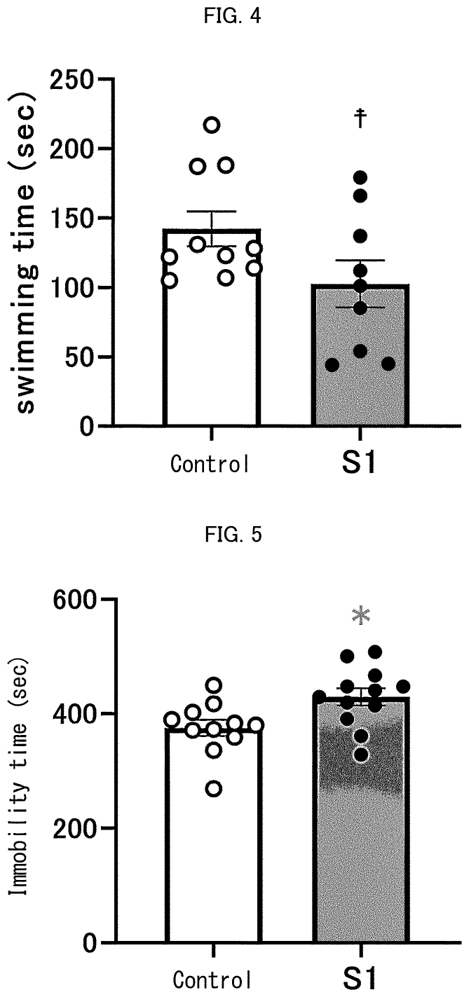

The results of the 10% weight-loaded forced swimming test are shown in FIG. 4. “Swimming time” on the vertical axis of the graph in FIG. 4 indicates the time (seconds) from when the mouse was placed in the water tank for the forced swimming test until the nose remained submerged below the water surface for 10 seconds. “Control” on the horizontal axis of the graph in FIG. 4 indicates the control mice, and “S1” indicates the S1 mice. ‡: P<0.1.

In comparison with the control mice, the S1 mice tended to have shorter swimming times. This indicates that the S1 mice are more prone to fatigue in comparison with the control mice.

### Example 3

### Expression of Depressive Disorder-Like Behavior in S1 Mice

### Method

The S1 mice and the control mice were prepared using the same method as discussed in Example 2. To confirm depressive disorder-like behavior, a tail suspension test was conducted. As the method, on the 6th day after nasal administration of S1/Adv or vector/Adv, the tails of the S1 mice and the control mice were fixed, and the mice were suspended for 10 minutes. The behavior was recorded, analysis was made using the image analysis software TailSuspScan (CleverSys Inc.), and the immobility time was measured.

### Results

The results of plotting the immobility time in the tail suspension test are shown in FIG. 5. “Control” on the horizontal axis of the graph in FIG. 5 indicates the control mice, and “S1” indicates the S1 mice. *: P<0.05.

In comparison with the control mice, the S1 mice showed a significant increase in immobility time. This revealed that the S1 mice exhibit depressive disorder-like behavior.

### Example 4

Damage to olfactory bulb nerves in mice in which S1 protein is expressed and inflammation is induced by LPS

### Method

The S1 mice and the control mice were prepared using the same method as discussed in Example 2. On the 7th day after nasal administration of S1/Adv or vector/Adv, the S1 mice and the control mice were abdominally administered with lipopolysaccharide (MERCK) derived from E. coli O111 at a dose of 5 mg/kg. The olfactory bulbs were collected 30 minutes and 60 minutes later.

Using the collected olfactory bulbs, RNA was purified with the RNeasy Mini Kit (QIAGEN). The purified RNA was used as a template to synthesize cDNA with the PrimeScript RT reagent Kit (Takara-Bio Inc.). Using the synthesized cDNA, the gene expression of calbindin was analyzed by RT-qPCR. The measurement results of 18S rRNA were used for normalization.

### Results

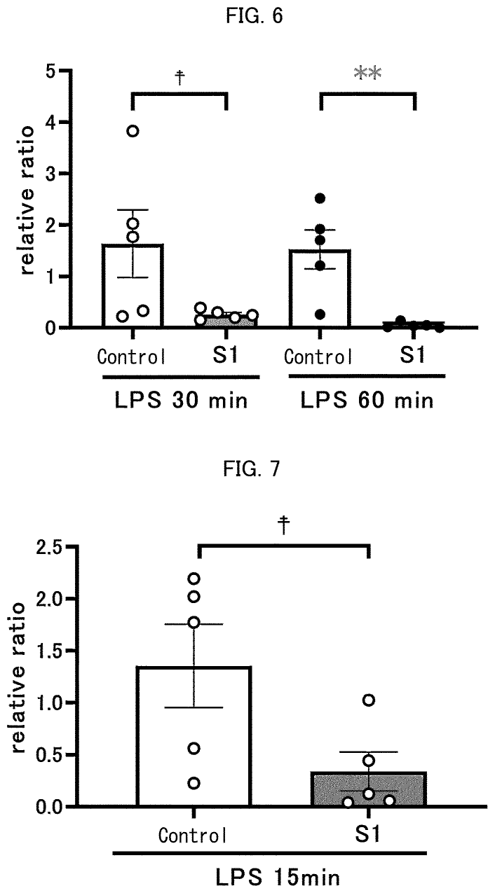

The results of the RT-qPCR analysis are shown in FIG. 6. “Control” on the horizontal axis of the graph in FIG. 6 indicates the control mice, and “S1” indicates the S1 mice. ‡: P<0.1, **: P<0.01.

The results of the RT-qPCR analysis revealed that the expression level of calbindin in the olfactory bulbs of the S1 mice tended to decrease 30 minutes after the LPS administration, and significantly decreased 60 minutes after the LPS administration. Since calbindin is a marker of mature neurons, it is considered that when peripheral inflammation is induced, the mature neurons in the olfactory bulbs of the S1 mice are damaged.

### Example 5

Damage to cranial nerves in mice in which S1 protein is expressed and inflammation is induced by LPS

### Method

The S1 mice and the control mice were prepared using the same method as discussed in Example 2. On the 7th day after nasal administration of S1/Adv or vector/Adv, the S1 mice and the control mice were abdominally administered with lipopolysaccharide (MERCK) derived from E. coli O111 at a dose of 5 mg/kg. The brains excluding the olfactory bulbs were collected 15 minutes later.

Using the collected brains, RNA was purified with the RNeasy Mini Kit (QIAGEN). The purified RNA was used as a template to synthesize cDNA with the PrimeScript RT reagent Kit (Takara-Bio Inc.). Using the synthesized cDNA, the gene expression of calbindin was analyzed by RT-qPCR. The measurement results of 18S rRNA were used for normalization.

### Results

The results of the RT-qPCR analysis are shown in FIG. 7. “Control” on the horizontal axis of the graph in FIG. 7 indicates the control mice, and “S1” indicates the S1 mice. ‡: P<0.1.

The results of the RT-qPCR analysis showed that the expression level of calbindin in the brains of the S1 mice tended to decrease 15 minutes after LPS administration. Therefore, it is considered that when peripheral inflammation is induced, the mature neurons in the brains of the S1 mice are damaged.

### Example 6

Damage to cholinergic neurons in mice in which S1 protein is expressed

### Method

On the 7th day after nasal administration of S1/Adv or vector/Adv, the brains of the S1 mice and the control mice were extracted and fixed in a 10% neutral formalin solution. The fixed brains were embedded in paraffin, and coronal sections (brain sections) were prepared at a position where medial septum and diagonal band of Broca can be observed (Bregma 0.62 mm). The prepared brain sections were deparaffinized, subjected to antigen activation treatment, and then fluorescently immunostained with an Anti-Choline Acetyltransferase antibody (manufactured by abcam).

### Results

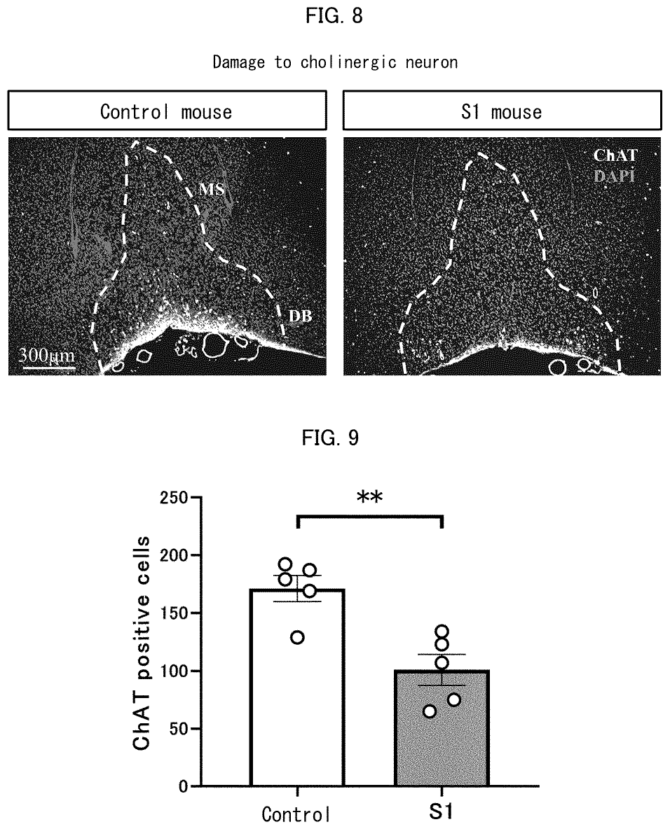

The results of the fluorescent immunostaining are shown in FIG. 8. FIG. 8 suggests that in the S1 mice, compared with the control mice, the number of cholinergic neurons, which are choline acetyltransferase (ChAT) positive cells in the medial septum (MS) and the diagonal band (DB) of Broca of the basal forebrain, was decreased. Therefore, the number of ChAT-positive cells in the MS/DB region was measured. The results are shown in FIG. 9. FIG. 9 reveals that in the S1 mice, the number of ChAT-positive cells in the MS/DB region was significantly decreased. **: P<0.01.

These results indicate that cholinergic neurons in the brains of the S1 mice were impaired. Therefore, a possibility was suggested that the amount of acetylcholine in the brains of the S1 mice is decreased.

### Example 7

### Improvement of Fatigue Symptoms in S1 Mice by Donepezil Administration

### Method

### Administration of Donepezil

Donepezil (FUJIFILM Wako Pure Chemical Corporation) was dissolved in water at 32 mg/L and administered to the mice via drinking water. This resulted in a donepezil dosage of 4.0 mg/kg/day for the mice. The dosage of 4.0 mg/kg/day was employed because a large number of literature disclose donepezil dosages of 3.0 mg/kg/day to 5.0 mg/kg/day.

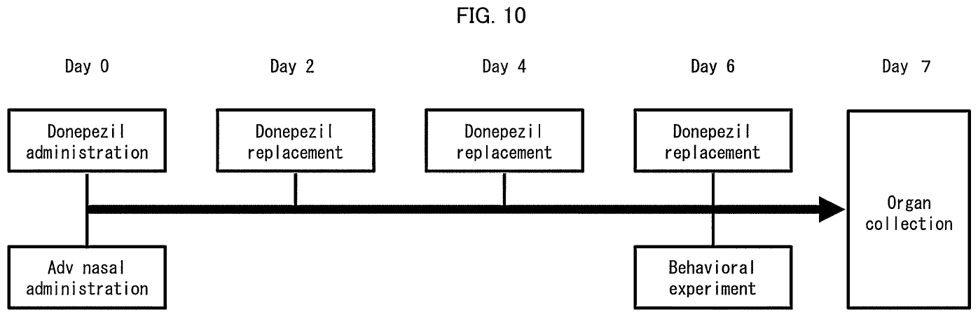

FIG. 10 shows administration scheme of donepezil. The donepezil solution was administered to the S1 mice and the control mice immediately after the nasal administration of S1/Adv or vector/Adv, and the drinking water containing donepezil was replaced every two days. It should be noted that donepezil was administered in the same manner in the following Examples.

### Fatigue Behavioral Test

On the 6th day after nasal administration of S1/Adv or vector/Adv, a 10% weight-loaded forced swimming test was conducted as a fatigue behavioral test. As the method, the body weights of the S1 mice and the control mice were measured in the morning of the test day, and weights corresponding to approximately 10% of the body weights of the mice were prepared. The weights were fixed to the tails of the S1 mice and control mice, and the mice were placed in a water tank for the forced swimming test. The period of time until the nose of the mouse was submerged below the water surface for 10 seconds was measured.

### Results

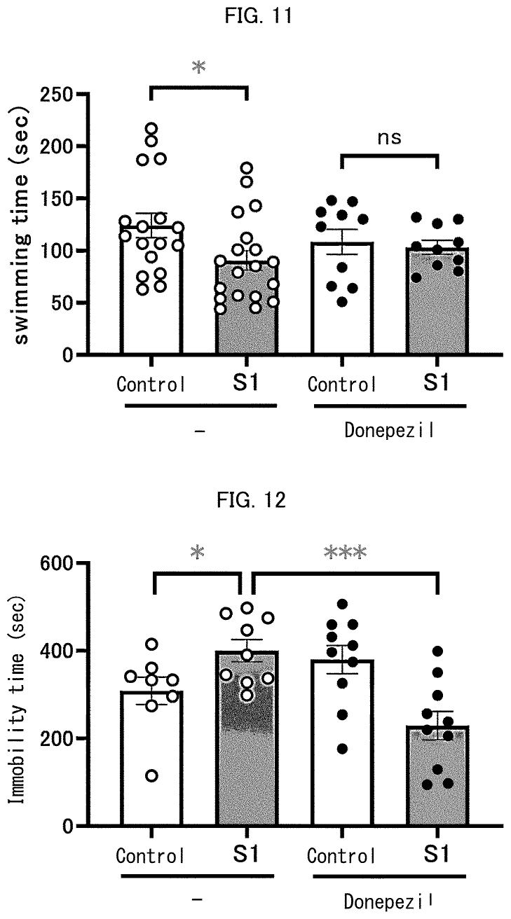

The results of the 10% weight-loaded forced swimming test are shown in FIG. 11. “Swimming time” on the vertical axis of the graph in FIG. 11 indicates the time (seconds) from when the mouse was placed in the water tank for the forced swimming test until the nose remained submerged below the water surface for 10 seconds. “Control” on the horizontal axis of the graph in FIG. 11 indicates the control mice, and “S1” indicates the S1 mice. *: P<0.05.

In the group not administered with donepezil (indicated as “-” in FIG. 11), the swimming time of the S1 mice was significantly shorter in comparison with the control mice. In contrast, in the group administered with donepezil (indicated as “Donepezil” in FIG. 11), no difference was observed in the swimming time between the control mice and the S1 mice. This result is considered to suggest that donepezil improves the fatigue-like behavior induced by the expression of the S1 protein.

### Example 8

### Improvement of Depressive Disorder-Like Behavior in S1 Mice by Donepezil Administration

### Method

For the S1 mice and the control mice that were administered through drinking water with donepezil according to the administration scheme illustrated in FIG. 10 after nasal administration of S1/Adv or vector/Adv, a tail suspension test was conducted to confirm depressive disorder-like behavior. As the method, on the 6th day after nasal administration of S1/Adv or vector/Adv, the tails of the S1 mice and the control mice were fixed, and the mice were suspended for 10 minutes. The behavior was recorded, analysis was made using the image analysis software TailSuspScan (CleverSys Inc.), and the immobility time was measured.

### Results

The results of plotting the immobility time in the tail suspension test are shown in FIG. 12. “Control” on the horizontal axis of the graph in FIG. 12 indicates the control mice, and “S1” indicates the S1 mice. *: P<0.05, ***: P<0.001.

In the group not administered with donepezil (indicated as “-” in FIG. 12), the immobility time of the S1 mice was significantly increased in comparison with the control mice. In contrast, in the group administered with donepezil (indicated as “Donepezil” in FIG. 12), the immobility time in the S1 mice was significantly decreased in comparison with the control mice. This result is considered to suggest that donepezil improves the depressive disorder-like behavior induced by the expression of the S1 protein.

### Example 9

### Improvement of Brain Inflammation in S1 Mice by Donepezil Administration

### Method

From the S1 mice and the control mice that were administered through drinking water with donepezil according to the administration scheme illustrated in FIG. 10 after nasal administration of S1/Adv or vector/Adv, the brains excluding the olfactory bulbs were collected on the 7th day after the nasal administration of S1/Adv or vector/Adv. Using the collected brains, RNA was purified with the RNeasy Mini Kit (QIAGEN). The purified RNA was used as a template to synthesize cDNA with the PrimeScript RT reagent Kit (Takara-Bio Inc.). Using the synthesized cDNA, the gene expression of interleukin-6 (IL-6), tumor necrosis factor (TNFα), and chemokine CC motif ligand 2 (CCL2) was analyzed by RT-qPCR. The measurement results of 18S rRNA were used for normalization.

### Results

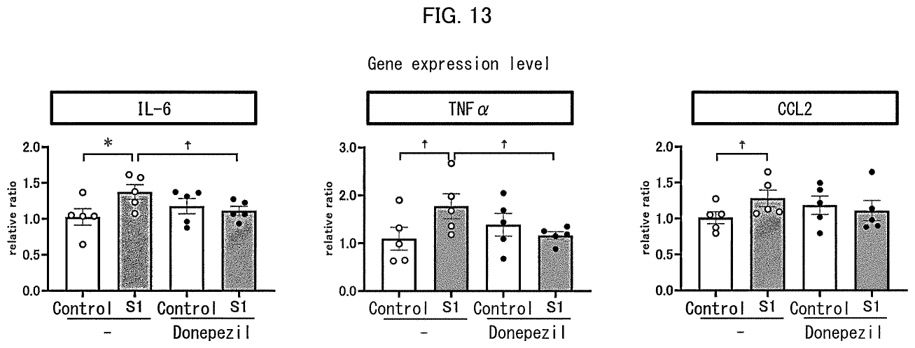

The results of the RT-qPCR analysis are shown in FIG. 13. “Control” on the horizontal axis of the graph in FIG. 13 indicates the control mice, and “S1” indicates the S1 mice. *: P<0.05, ‡: P<0.1.

The results of the RT-qPCR analysis revealed that in the group not administered with donepezil (indicated as “-” in FIG. 13), the expression of IL-6 in the brains of the S1 mice was significantly increased, and the expression of TNFα and CCL2 also exhibited a tendency to increase. In contrast, in the group administered with donepezil (indicated as “Donepezil” in FIG. 13), the expression of these genes in the brains of the S1 mice tended to decrease, and no difference was observed in comparison with the expression of these genes in the brains of the control mice. Therefore, it was revealed that brain inflammation is induced in the S1 mice due to the expression of the S1 protein, and the brain inflammation is improved by the administration of donepezil.

### Example 10

### Evaluation of Effect of Acetylcholine Receptor Agonists other than Donepezil

### Method

Donepezil exerts its effect by increasing the amount of acetylcholine in the brain. Therefore, to develop more effective therapeutic agents, it is necessary to perform screening using a drug that more selectively acts on acetylcholine receptors. Therefore, to the S1 mice and the control mice, on the 7th day after nasal administration of S1/Adv or vector/Adv, PNU282987, which is an agonist specific to the a7 nicotinic acetylcholine receptor, was administered intracerebroventricularly at a dose of 400 nmol/mouse, and the therapeutic effect on brain inflammation in the S1 mice was examined.

One hour after the intracerebroventricular administration of PNU282987, the brains excluding the olfactory bulbs were collected, and RNA was purified with the RNeasy Mini Kit (QIAGEN). The purified RNA was used as a template to synthesize cDNA with the PrimeScript RT reagent Kit (Takara-Bio Inc.). Using the synthesized cDNA, the gene expression of interleukin-1β (IL-1β) and interleukin-6 (IL-6) was analyzed by RT-qPCR. The measurement results of 18S rRNA were used for normalization.

### Results

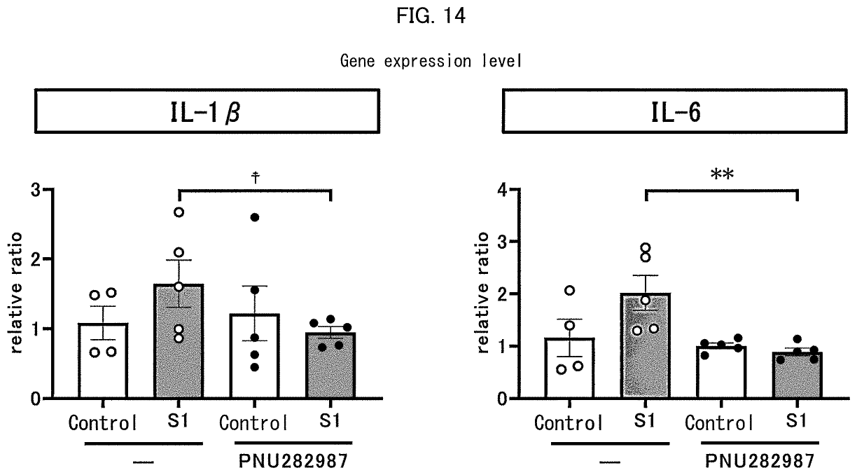

The results of the RT-qPCR analysis are shown in FIG. 14. “Control” on the horizontal axis of the graph in FIG. 14 indicates the control mice, and “S1” indicates the S1 mice. **: P<0.01, ‡: P<0.1. The increased expression levels of inflammatory cytokine genes (IL-1ß and IL-6) in the brain, which are considered as the molecular mechanisms of fatigue and depression observed in S1 mice, were suppressed by intracerebroventricular administration of PNU282987. These results are considered to suggest that PNU282987 and derivatives thereof can be potential candidates for therapeutic agents for brain inflammation in S1 mice, and that α7 nicotinic acetylcholine receptor agonists are the drugs which should be prioritized in screening for therapeutic agents.

### Example 11

### Examples of Mechanism of Action of Acetylcholine Receptor Agonists

### Method

To elucidate the molecular mechanism of the therapeutic effect of PNU282987 on brain inflammation in Example 10, the impact of S1 and dPNU282987 on the gene expression level of zinc finger protein 36 (Zfp36), which is a host protein with anti-inflammatory effects, was examined. Specifically, using the synthesized cDNA in Example 10, the gene expression of Zfp36 was analyzed by RT-qPCR. The measurement results of 18S rRNA were used for normalization.

### Results

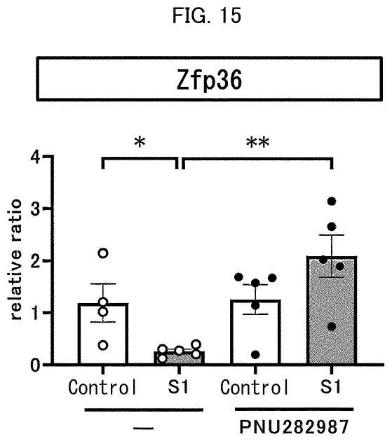

The results of the RT-qPCR analysis are shown in FIG. 15. “Control” on the horizontal axis of the graph in FIG. 15 indicates the control mice, and “S1” indicates the S1 mice. *: P<0.05, **: P<0.01. These results revealed that S1 mice exhibited a decrease in ZFP36, and this decrease in ZFP36 was improved by the administration of PNU282987. This suggests that brain inflammation in S1 mice is due to the decrease in ZFP36, which has anti-inflammatory action, and that acetylcholine receptor agonists, particularly the α7 nicotinic acetylcholine receptor agonists, exert therapeutic effects on brain inflammation by restoring the expression level of ZFP36. These results are considered to indicate that ZFP36 can be a therapeutic agent target and that the expression level of ZFP36, which is the gene thereof, can serve as a biomarker for therapeutic agent screening.

From the above results, it was revealed that COVID-19 sequela model mouse can be prepared by expressing the S1 protein in mice, that the cholinergic neurons in the brain of the COVID-19 sequela model mouse are impaired, and that the symptoms of COVID-19 sequelae in the COVID-19 sequela model mouse can be improved by the administration of donepezil. In addition, from the results of analyzing the mechanism of action of acetylcholine receptor agonists, it was revealed that, among acetylcholine receptor agonists, α7 nicotinic acetylcholine receptor agonists are the drugs that should be prioritized in screening for therapeutic agents for COVID-19 sequelae.

### INDUSTRIAL APPLICABILITY

The COVID-19 sequela therapeutic agent in accordance with an aspect of the present invention can contribute to the treatment or prevention of COVID-19 sequelae in patients infected with the novel coronavirus. The COVID-19 sequela therapeutic agent screening method in accordance with an aspect of the present invention can contribute to the development of new therapeutic agents for COVID-19 sequelae. The COVID-19 sequela model animal produced by the COVID-19 sequela model animal production method in accordance with an aspect of the present invention can be used in the development of pharmaceutical products, the basic research on COVID-19 sequelae, and the like.

1. (canceled)

2. (canceled)

3. (canceled)

4. (canceled)

5. (canceled)

6. (canceled)

7. (canceled)

8. A method for screening a therapeutic agent for a sequela of a novel coronavirus infection, said method comprising:
administering a test substance into a novel coronavirus infection sequela model animal obtained by expressing SARS-COV-2 S1 protein in a non-human mammal; and
evaluating a change in a novel coronavirus-related symptom of the model animal before and after administration of the test substance.

9. The method according to claim 8, wherein the model animal is a model animal obtained by expressing SARS-COV-2 S1 protein in at least one of a nasal cavity and a part around the nasal cavity of the non-human mammal.

10. The method according to claim 8 or 9, wherein the model animal is a model animal produced by a novel coronavirus infection sequela model animal production method including expressing SARS-COV-2 S1 protein in the non-human mammal with use of a SARS-COV-2 S1 protein expression vector.

11. The method according to claim 10, wherein the novel coronavirus infection sequela model animal production method further includes inducing inflammation in the non-human mammal.

12. A method for producing a novel coronavirus infection sequela model animal, comprising expressing SARS-COV-2 S1 protein in a non-human mammal with use of a SARS-COV-2 S1 protein expression vector.

13. The method according to claim 12, wherein, in the expressing, the SARS-CoV-2 S1 protein is expressed in at least one of a nasal cavity and a part around the nasal cavity of the non-human mammal.

14. The method according to claim 12, further comprising inducing inflammation in the non-human mammal.

15. The method according to any one of claims 12 through 14 claim 12, wherein the SARS-COV-2 S1 protein is one of the following polypeptides (a) and (b):
(a) a polypeptide that has an amino acid sequence represented by SEQ ID NO: 1; and
(b) a polypeptide that is constituted by an amino acid sequence having a sequence identity of 80% or more to the amino acid sequence represented by SEQ ID NO: 1 and that has activity to raise an intracellular calcium concentration when introduced into a cell.

16. A method for treating or preventing a sequela of a novel coronavirus infection, said method comprising administering, into a subject, a novel coronavirus infection sequela therapeutic agent containing an acetylcholine receptor agonist as an active ingredient.

17. The method according to claim 16, wherein the acetylcholine receptor agonist is a central acetylcholine receptor agonist that acts on an intracerebral acetylcholine receptor.

18. The method according to claim 16, wherein the acetylcholine receptor agonist is donepezil.

19. The method according to claim 16, wherein the sequela of the novel coronavirus infection is novel coronavirus-related fatigue.

20. The method according to claim 16, wherein the sequela of the novel coronavirus infection is a novel coronavirus-related depressive symptom.

21. The method according to claim 16, wherein the sequela of the novel coronavirus infection is a novel coronavirus-related olfactory dysfunction.

22. The method according to claim 16, wherein the sequela of the novel coronavirus infection is a novel coronavirus-related memory impairment.
